(19) United States
(12) Patent Application Publication
(10) Pub. No.: US 2025/0298008 A1
(43) Pub. Date: Sep. 25, 2025

(54) DUAL-SYSTEM METHOD FOR ASSESSING TRANSMISSIBILITY AND DISEASE SEVERITY OF RESPIRATORY VIRUSES

(71) Applicant: Centre for Immunology & Infection Limited, Hong Kong (HK)

(72) Inventors: Chi Wai Michael CHAN, Hong Kong (HK); Pui Yan HUI, Hong Kong (HK); John Malcolm NICHOLLS, Hong Kong (HK); Joseph Sriyal Malik PEIRIS, Hong Kong (HK)

(21) Appl. No.: 19/082,225

(22) Filed: Mar. 18, 2025

Related U.S. Application Data
(60) Provisional application No. 63/569,229, filed on Mar. 25, 2024.

(51) Int. Cl. G01N 33/50; C12N 5/071; C12Q 1/06; G01N 21/64

(52) U.S. Cl. CPC G01N 33/5091; C12N 5/0688; C12Q 1/06; G01N 21/6408; G01N 21/6486; G01N 33/5044; G01N 2333/075; G01N 2333/095; G01N 2333/11; G01N 2333/165

(57) ABSTRACT

The present invention uses ex vivo human airway cultures to assess the human transmissibility and replication competence of influenza and coronavirus strains. By comparing pandemic influenza A subtype H1N1 and highly pathogenic avian influenza H5N1 as reference strains, the transmissibility risk of various viruses was evaluated and categorized. Additionally, an in vitro model evaluated virus-induced impairment of alveolar fluid clearance (AFC) as an indicator of disease severity. The study revealed correlations between bronchus viral replication, human transmission, AFC impairment, and clinical disease severity across different influenza and coronavirus strains.

### CROSS-REFERENCE TO RELATED APPLICATIONS

The present application claims the priorities from the U.S. provisional patent application Ser. No. 63/569,229 filed Mar. 25, 2024, and the disclosure of which is incorporated herein by reference in its entirety.

### FIELD OF THE INVENTION

The present invention generally relates to the evaluation and assessment of viral infections. This encompasses aspects such as the replication capacity of viruses within the human respiratory system, their impact on lung tissues, and the assessment of disease severity associated with these infections.

### BACKGROUND OF THE INVENTION

Pandemics can emerge unexpectedly and spread rapidly, posing significant health risks before vaccines become available. To mitigate these risks, global surveillance programs monitor animal influenza viruses circulating in wild birds, poultry, and swine. However, a major gap exists in accurately assessing the risk of these animal viruses becoming transmissible among humans. Current tools, such as the CDC's Influenza Risk Assessment Tool (IRAT) and WHO's Tool for Influenza Pandemic Risk Assessment (TIPRA), evaluate viruses based on emergence risk and public health impact but require further refinement in key parameters.

One crucial parameter with heavy weighting for assessing the risk of a virus acquiring transmissibility in humans is its ability to infect and replicate in the human upper respiratory tract. Currently, this is largely assessed indirectly by examining virus binding to sialic acids linked to galactose, which are assumed to reflect virus receptors on epithelial cells of the upper airways. However, the diversity and complexity of glycans in the human upper airways are not fully understood, and existing glycan arrays do not represent all relevant glycan structures. This limitation hinders accurate assessment of human receptor binding and virus transmissibility.

An alternative approach involves using ex vivo cultures of human airways to investigate whether a virus can infect and replicate in these epithelial cells. This method provides a more direct assessment compared to chemical binding assays and correlates well with airborne transmission in ferrets, a model for human transmissibility. However, previous methods using ex vivo cultures lacked reproducibility and quantitation. Additionally, donor variability presents challenges in consistency, and ex vivo lung cultures are inadequate for assessing disease severity, as both highly pathogenic avian influenza (HPAI) H5N1 and seasonal influenza viruses infect human lungs but cause differing levels of disease severity.

Current risk assessment tools mainly rely on animal models for transmission studies and chemical binding assays to evaluate receptor-virus binding affinity. However, these models do not accurately reflect human physiological conditions. Therefore, there is a critical need for new technologies that provide a physiologically relevant model to more accurately assess the risk of animal influenza viruses acquiring human transmissibility and causing severe disease.

### SUMMARY OF THE INVENTION

Emergence of animal influenza viruses circulating in the poultry and human population imposes a huge public threat. Current risk assessment tools that link the gap between surveillance and their risk in human transmission and disease severity are lacking.

Accordingly, the present invention provides a physiologically relevant model for assessing the pandemic potential and disease severity of pathogens (e.g., respiratory viruses). This model evaluates the impact of each virus on alveolar fluid clearance (AFC), a key indicator of disease severity, driven by soluble mediators in the virus supernatant rather than virus-induced cytopathic effects. This approach enables the correlation of disease severity between avian and human influenza viruses. Ultimately, this advancement enhances the risk assessment of human transmissibility and disease severity of influenza viruses, improving pandemic preparedness.

In one aspect, the present invention provides a dual-system method for assessing the human transmissibility and disease severity of respiratory viruses, including utilizing ex vivo human airway cultures to evaluate viral replication competence of the respiratory viruses in bronchial and lung tissues; utilizing an in vitro lung injury model to evaluate alveolar fluid clearance (AFC) impairment induced by the respiratory viruses to determine transmission risk; and correlating the viral replication competence in the bronchial and lung tissues with the relative human transmissibility score.

In one embodiment, the ex vivo human airway cultures include human bronchial tissues obtained from non-malignant lung resection surgeries.

In one embodiment, the viral replication competence is assessed by measuring viral titers in the culture supernatant at 1, 24, and 48 hours post-infection using a median tissue culture infectious dose assay.

In one embodiment, the method further includes normalizing area under the curve (AUC) of viral replication kinetic curves to reference strains to determine relative human transmissibility score.

In one embodiment, the reference strains include pandemic influenza A subtype H1N1 and highly pathogenic avian influenza A subtype H5N1.

In one embodiment, the in vitro lung injury model utilizes primary human alveolar epithelial cells (AECs) cultured on apical Transwell inserts to simulate the AFC.

In one embodiment, the AFC impairment is quantified by measuring changes in fluorescent intensity over a 24-hour infection period, and the AFC is calculated relative to mock-infected controls, with mock infections set as 100% AFC.

In one embodiment, the respiratory viruses include influenza A viruses, influenza B viruses, respiratory syncytial virus (RSV), adenoviruses, parainfluenza viruses, rhinoviruses, human metapneumovirus, enterovirus, or coronaviruses.

In one embodiment, the influenza A viruses include H1N1, H3N2, H5N1, H5N6, H5N8, H7N9, and H9N2 subtypes. The coronaviruses include SARS-CoV, MERS-CoV, and SARS-CoV-2.

In one embodiment, the method further includes comparing the AFC impairment of the respiratory virus to that of reference strains to categorize disease severity.

In one embodiment, the method further includes evaluating the impact of varying multiplicities of infection (MOIs) on the AFC impairment to assess dose-dependent pathogenicity.

In one embodiment, the MOIs range from 0.1 to 10.

Preferably, the representative MOIs is approximately 0.1.

In one embodiment, the AFC impairment is attributed to virus-induced soluble mediators rather than direct cytopathic effects.

In one embodiment, the method further includes calculating a relative disease severity score by comparing AFC impairment with reference treatment and strains.

In one embodiment, the dual-system method exhibits improved accuracy of quantification in disease severity compared to traditional ex vivo human airway cultures, showing 65.2%, 20.6% and 23.6% improvement in quantification accuracy in disease severity of seasonal influenza, avian influenza, and low pathogenic avian and influenza B viruses, respectively.

Unlike existing risk assessment tools, which rely on indirect evaluations such as virus binding to sialic acids or animal models that fail to accurately reflect human physiological conditions, this invention directly assesses viral replication and pathogenicity using ex vivo human airway cultures and an in vitro lung injury model. The ex-vivo culture of human airway can be used to directly assess the replication of a virus in the human bronchial and lung tissues.

One significant problem addressed by this invention is the limitation of current surveillance methods, which lack accurate predictive power for human transmissibility and disease severity of zoonotic viruses. Traditional tools are based on indirect parameters and animal models that do not fully represent the complexity of human respiratory tract physiology, leading to gaps in pandemic preparedness. This invention overcomes these challenges by utilizing a more accurate model system that directly measures virus replication competence in human bronchial and lung tissues, as well as virus-induced impairment of AFC.

### BRIEF DESCRIPTION OF THE DRAWINGS

Embodiments of the invention are described in more details hereinafter with reference to the drawings, in which:

FIG. 1A shows AFC rates for various influenza and coronavirus infections in the in vitro lung injury model, with results normalized to mock infections (set as 100). FIG. 1B shows viral titers corresponding to AFC measurements, shown as AUC from 24-48 hpi (n≥3). The data are presented as dot plots with mean±SEM. FIG. 1C shows the impact of different viral strains and MOIs on AFC. Fluorescent reading is measured at 5 min and 16 hpi. AFC is calculated and presented as bar charts (n≥3, mean±SEM);

FIG. 2. shows the replication competence of influenza viruses in ex vivo human bronchus cultures, with viral titers measured at 1, 24 and 48 hours post-infection (hpi). The results are presented as the area under the curve (AUC), normalized to H1N1pdm (set as 100) and HPAI H5N1 (set as 0). Viral titers in the culture supernatant of infected tissues are measured at 1, 24 and 48 hpi using TCID₅₀ assay. The AUC is calculated from replication kinetic between 24-48 hpi (n≥3). Relative AUC is calculated by normalization to H1N1pdm (415742/09) set as 100 and HPAI H5N1 (483/97) set as 0 and presented as bar charts with mean±SEM;

FIG. 3 shows the replication competence of influenza viruses in ex vivo human lung tissues, with viral titers measured at 1, 24, and 48 hpi. AUC values from 24-48 hpi (n≥3) are presented as bar charts with mean±SEM.

### DETAILED DESCRIPTION

Assessing the potential severity of an animal virus if it acquires transmissibility in humans is an important second dimension of pandemic risk assessment. There is greater incentive to invest in counter-measures against a potential pandemic of greater severity than for a milder one.

Presently, disease severity is assessed primarily from severity of human zoonotic disease. The pitfall is that only more severe zoonotic infections are likely to be recognized, thus skewing case reports towards greater severity. Current risk assessment tools, such as the CDC's IRAT and WHO's TIPRA, rely heavily on indirect parameters, including virus binding affinity to sialic acids and animal models that do not fully represent human respiratory physiology.

Tradition models exhibit several limitations, including:

### (1) Indirect Assessments and Limited Predictive Power

Existing models infer human transmissibility primarily through chemical binding assays that assess virus binding to glycans. However, the diversity and complexity of human airway glycans are not fully understood, limiting the predictive accuracy for human receptor binding and viral transmissibility. Furthermore, animal models, such as ferrets and mice, fail to accurately reflect human respiratory physiology and immune responses, leading to discrepancies between animal study results and human clinical outcomes.

### (2) Lack of Reproducibility and Quantitative Analysis

Current ex vivo cultures used to study viral replication are often hindered by donor variability and a lack of standardization, resulting in inconsistent and non-quantitative data. In addition, existing methods primarily focus on measuring viral titers without assessing functional outcomes, such as lung injury or AFC.

Accordingly, the present invention provides a novel and physiologically relevant model for evaluating the human transmissibility and disease severity of respiratory viruses by utilizing both ex vivo human airway cultures and an in vitro lung injury model.

The present invention describes the importance of measuring both the viral replication in bronchial tissues and the damage of the lung function by virus infection for a comprehensive assessment of viral pathogenicity; and describes a quantitative approach on analyzing virus replication, virus tropism and damage of lung function.

Notably, the present invention demonstrates that high viral replication in lung tissues does not necessarily correlate with severe lung damage, challenging the conventional reliance on replication titers as a sole measure of pathogenicity.

First, the present invention provides a physiologically-relevant in vitro lung injury model. The model assesses virus-induced impairment of AFC, which is driven by soluble mediators rather than cytopathic effects. Such approach allows for the correlation of disease severity between different virus strains, including avian and human influenza viruses.

Second, the present invention provides ex vivo human airway cultures used to directly risk assess the adaptation of a virus to transmit between humans, and further provided a semi-quantitative approach to evaluate influenza and coronavirus replication competence. The use of ex vivo human airway cultures eliminates the species-specific differences seen in animal models, providing a more accurate representation of human respiratory conditions. Moreover, normalizing AUC scores to reference strains reduces variability associated with donor differences, ensuring consistent and reliable results.

This dual-system approach can direct assessment of human viral replication and pathogenicity. Unlike existing tools, this invention directly measures viral replication in human bronchial and lung tissues. Additionally, the in vitro lung injury model assesses AFC impairment caused by virus-induced soluble mediators, rather than cytopathic effects, offering a more comprehensive assessment of disease severity.

By using human airway tissues and a semi-quantitative approach for AUC normalization against reference strains, this model minimizes donor variability and enhances reproducibility. The quantitative analysis of both viral replication and AFC impairment allows for more precise categorization of human transmissibility and disease severity compared to traditional models reliant on viral titers alone.

Moreover, a notable feature of the present invention is its capability to assess viral replication and AFC impairment at lower MOIs compared to existing technologies. The in vitro lung injury model demonstrates sensitivity to detect AFC impairment at MOIs as low as 0.1, whereas conventional models typically require higher MOIs to observe similar effects. This enhanced sensitivity not only improves the accuracy of pathogenicity assessments but also reflects more physiologically relevant viral loads observed in human infections.

In one embodiment, the present invention is employed to evaluate various viruses, including influenza A viruses, influenza B viruses, respiratory syncytial virus (RSV), adenoviruses, parainfluenza viruses, rhinoviruses, human metapneumovirus, enterovirus, or coronaviruses.

In one embodiment, the viruses include seasonal H1N1 and H3N2 viruses known for efficient human-to-human transmission, zoonotic HPAI H5N1, H5N6, H7N9 and H9N2 as well as other HPAI H5N8 and LPAI viruses not associated with zoonotic disease. Using pandemic H1N1 and HPAI H5N1 viruses as reference strains of proven human transmissibility and lack of transmissibility respectively, a relative score of tested viruses to transmit between humans is calculated.

Seasonal influenza H1N1, H3N2, influenza B and MERS-CoV have productive bronchus viral replication and tissue infection, but minimal for wild bird surveillance isolates H5N3 and H7N1 when referenced to pandemic H1N1 and HPAI H5N1. Differential patterns of lung viral replication are detected for H5N6 and H9N2. HPAI H5N1, H7N9 and SARS-CoV have more severe AFC impairment than seasonal H1N1, H3N2 and influenza B viruses which correlate with their respective clinical observation of disease severity.

By comparing test viruses to well-characterized reference strains-pandemic H1N1 (with proven human transmissibility) and HPAI H5N1 with minimal human transmissibility, the invention provides a relative score for human transmission risk.

One of the most unexpected and technically significant findings of this invention is the moderately negative correlation observed between viral replication and AFC impairment. This result challenges the conventional pathogenicity paradigm, which traditionally associates high viral replication with severe disease outcomes. By demonstrating that severe AFC impairment is mediated by soluble factors rather than direct cytopathic effects, the present invention provides new insights into the pathogenesis of respiratory viruses. This discovery not only advances the understanding of virus-induced lung injury but also underscores the necessity of evaluating both viral replication and functional impairment to obtain a comprehensive assessment of disease severity. This paradigm shift highlights the superiority of the present invention over conventional models that rely solely on viral titers as a measure of pathogenicity.

The findings convey an important association between viral replication and human transmission in ex vivo explants as well as the impairment of alveolar fluid clearance in vitro and clinical disease manifestation of different influenza virus and coronavirus strains.

### EXAMPLE

### Example 1

### Materials

Influenza viruses and coronaviruses used in the present invention and their virus isolation origin are listed in Table 1. The virus strains, abbreviation, subtypes and virus isolation origin of influenza A, influenza B viruses and coronaviruses are listed.

Wild bird fecal samples are collected during routine surveillance at the Hong Kong Mai Po.

All influenza viruses are passaged in Madin-Darby Canine Kidney (MDCK) whereas coronaviruses are grown in Vero E6 or MRC-5 cells. Viral titers are determined by median tissue culture infectious dose (TCID₅₀). All experiments are performed inside a biosafety level-3 facility.

TABLE 1
influenza viruses and coronaviruses evaluated for replication competence in ex vivo
explant infection and disease severity in the in vitro human lung injury model

| Virus Strain | | | |
|---|---|---|---|
| Influenza A | Abbreviation | Subtype | Virus isolation origin |
| A/Hong Kong/54/1998 | H1N1 (54/98) | H1N1 | Human |
| A/Oklahoma/447/2008 | H1N1 (447/08) | H1N1 | Human |
| A/Hong Kong/415742/2009 | H1N1pdm (415742/09) | H1N1pdm | Human |
| A/Hong Kong/1174/1999 | H3N2 (1174/99) | H3N2 | Human |
| A/Oklahoma/1992/2005 | H3N2 (1992/05) | H3N2 | Human |
| A/Hong Kong/483/1997 | H5N1 (483/97) | H5N1 | Human |
| A/Vietnam/1203/2004 | H5N1 (1203/04) | H5N1 | Human |
| A/Shenzhen/1/2012 | H5N1 (SZ1/12) | H5N1 | Human |
| A/Hong Kong/MPQ1017/2015* | H5N3 (MPQ1017/15) | H5N3 | Wild bird/Avian |
| A/Guangzhou/39715/2014 | H5N6 (39715/14) | H5N6 | Human |
| A/Oriental magpie robin/Hong Kong/6154/2015 | H5N6 (6154/15) | H5N6 | Wild bird/Avian |
| A/Northern pintail/Hong Kong/MP5883/2004 | H5N8 (MP5583/04) | H5N8 | Duck/Avian |
| A/Hong Kong/MPQ1219/2015* | H7N1 (MPQ1219/15) | H7N1 | Wild bird/Avian |
| A/Shanghai/1/2013 | H7N9 (Sh1/13) | H7N9 | Human |
| A/Shanghai/2/2013 | H7N9 (Sh2/13) | H7N9 | Human |
| A/Anhui/1/2013 | H7N9 (AH1/13) | H7N9 | Human |
| A/Qingyuan/GIRD1/2017 | H7N9 (QY/17) | H7N9 | Human |
| A/Quail/Hong Kong/G1/1997 | H9N2 (G1/97) | H9N2 | Quail/Avian |
| A/Duck/Hong Kong/Y280/1997 | H9N2 (Y280/97) | H9N2 | Duck/Avian |
| Influenza B | | Lineage | |
| B/Hong Kong/407373/2011 | B (407373/11) | Victoria | Human |
| B/Hong Kong/448799/2012 | B (448799/12) | Yamagata | Human |
| Coronavirus | | Clade | |
| HCoV-EMC/2012 | MERS-CoV | Prototype | Human |
| HK39849 | SARS-CoV | | Human |
| hCoV-19/Hong Kong/WHV-HK61-P3/2020 | SARS-CoV-2 | A | Human |

*viruses isolated from avian surveillance at the Hong Kong Mai Po Natural Reserve

### Methods

### Auc Analysis of Viral Replication Competence in Ex Vivo Culture

Viral replication competence in ex vivo cultures of human bronchus and lung is presented as AUC by GraphPad Prism v5.0 (GraphPad Software, USA). AUC is determined between virus replication kinetic curves and the detection limit of TCID₅₀ assay (10^{1.5}) at 24 and 48 hpi using Prism. For ex vivo bronchus culture, AUC of reference strain pandemic H1N1 (A/Hong Kong/415742/2009) is set as 100 and HPAI H5N1 (A/Hong Kong/483/1997) as 0 for normalization of each test virus.

### Statistical Analyses

Ex vivo infections and AFC of multiple viruses are compared using one-way ANOVA with Bonferroni post-tests in Prism.

### Example 2

### Establishment of In Vitro Lung Injury Model

Primary human alveolar epithelial cells (AECs) on apical Transwell inserts are infected with influenza viruses at MOIs of 0.1. After infection, cells are replenished with growth medium (SAGM™, Lonza, USA) containing 12.5 μg FITC-labeled dextran (Sigma). Mock-infected AECs are used as negative control. Net AFC is determined by change in fluorescent intensity of dextran over a 24 h-infection period and normalized to mock infection to define relative AFC. Viral titers of culture supernatant of AECs are measured at 1, 24 and 48 hpi by TCID₅₀ assay and AUC is calculated for 24-48 hpi using Prism. Relative AFC is calculated by normalizing to corresponding mock infection set as 100 and presented as dot plots with mean±SEM (n≥3). The relative AUC scores facilitate a more accurate categorization of human transmission risk and disease severity, enhancing pandemic risk assessment algorithms and public health decision-making.

To test the impact of MOI on AFC, AECs are infected with H1N1, H3N2 and H1N1pdm of MOI 0.1, 1 and 10, and HPAI H5N1 at MOI 0.1. AFC is measured at 16 hpi to minimize direct cytopathic effect on AFC since cell death observed with MOI 10 at 24 hpi.

### Example 3

### Evaluation of Alveolar Fluid Clearance as a Correlate of Disease Severity

This example illustrates the use of the in vitro lung injury model to evaluate AFC. as an indicator of disease severity for different influenza and coronavirus strains.

Previously studies using the in vitro lung injury model demonstrated that HPAI H5N1 (483/97) virus-infected AECs had impaired AFC, whereas seasonal H1N1 (54/98) virus had minimal impact on AFC, even though this virus replicated efficiently in AECs.

Moreover, clinically, HPAI H5N1 is associated with severe respiratory diseases, while H1N1 pandemic generally causes milder diseases but are more transmissible in humans. These observations underscore the necessity of evaluating both viral replication and lung function impairment for comprehensive assessment of disease severity.

This model provides a physiologically relevant approach by measuring AFC, which reflects lung function impairment caused by virus-induced soluble mediators rather than direct cytopathic effects.

To establish a comparative framework, HPAI H5N1 (483/97) and H1N1 pandemic (415742/09) viruses are used as reference strains due to their distinct clinical disease severity profiles. The H5N1 virus is known to cause severe lung damage with low AFC, whereas the H1N1 pandemic virus induces milder lung disease with high relative AFC. By comparing test viruses to these reference strains, the relative AFC scores are calculated to determine the potential severity of lung injury.

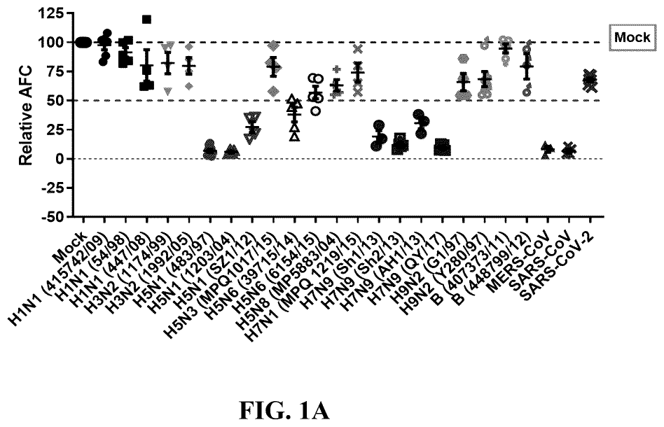

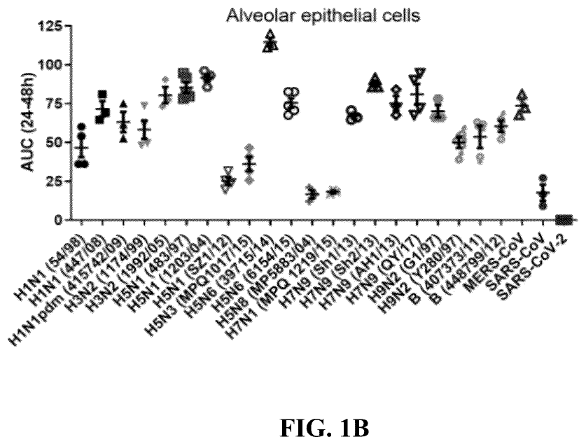

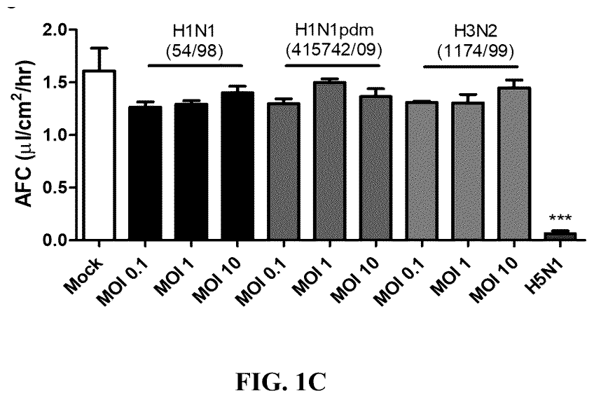

FIG. 1A shows AFC rates induced by various influenza viruses. Seasonal H1N1, H3N2, H5N3, and influenza B viruses exhibit AFC similar to that of H1N1 pandemic virus (>78% relative AFC). In contrast, H5N1 (SZ1/12), H5N6, H5N8, H7N1 and H9N2 viruses have moderate relative AFC score of around 25-75% whereas A/H5N1 (1203/04), H7N9, MERS-CoV and SARS-CoV have low relative AFC closer to HPAI H5N1 (483/97) around ≤25% relative AFC. These HPAI H5N1 and H7N9 viruses have known to cause more severe lung disease than the other viruses.

FIG. 1B illustrates the relationship between viral replication and AFC in the in vitro lung injury model. There is moderately negative correlation between viral replication (indicated by AUC) and AFC index. All the viruses replicate at least 1 log above the detection limit (0.15 AUC or 10^{1.5} TCID₅₀/mL). H3N2 (1992/05), H5N1 (SZ1/12), H5N8, H7N1 and B (448799/12) have relatively low replication, less than half the level of replication of other viruses.

To determine the impact of MOI on AFC in the in vitro model, AECs are infected with seasonal H1N1 (54/98 and 1174/99) and pandemic H1N1 (415742/09) viruses at increasing MOI from 0.1 to 10. Referring to FIG. 1C, AFC is not changed with increased viral titers at 16 hpi when compared to that of H5N1 (483/97) virus at MOI 0.1. Therefore, AFC is not affected by different MOI.

The moderately negative correlation between viral replication (AUC) and AFC index underscores the complex relationship between virus replication competence and lung injury. This finding challenges the traditional view that higher replication automatically leads to greater pathogenicity, emphasizing the role of virus-induced soluble mediators in disrupting AFC. These results highlight the importance of evaluating both viral replication and lung function impairment to gain a comprehensive understanding of disease severity.

### Example 4

### Establishment of Ex Vivo Human Bronchus Cultures

Human bronchus tissues were obtained from patients undergoing lung resection surgery with informed consent. Patient consents were obtained and the study was approved by the Institutional Review Board of the University of Hong Kong and Hospital Authority (approval no: UW 14-119).

Fresh, non-malignant bronchus tissues are processed post-surgery. Tissues are cut into small pieces (around 1-5 mm³) and washed with phosphate-buffered saline (PBS) containing antibiotics. Tissue fragments are placed on a transwell insert with the epithelial surface facing up. The basal chamber is filled with culture medium to maintain an air-liquid interface, mimicking the natural respiratory environment. The cultures are incubated at 37° C. with 5% CO₂. After 48 hours, tissues are infected with influenza viruses. Ciliated and non-ciliated epithelial cells infectivity are assessed by a clinical pathologist.

This ex vivo human bronchus culture system provides a physiologically relevant model for assessing viral replication and human transmissibility, offering a more accurate evaluation compared to traditional cell lines or animal models.

### Example 5

### Replication Competence of Influenza Viruses and Coronaviruses in Human Bronchus

AUC is defined by the area between detection limit and the replication kinetic curve at 24 and 48 hpi. Seasonal influenza A and B viruses exhibit substantial replication on ex vivo human bronchus cultures with relative mean AUC 241.1 (minimal AUC index of seasonal viruses).

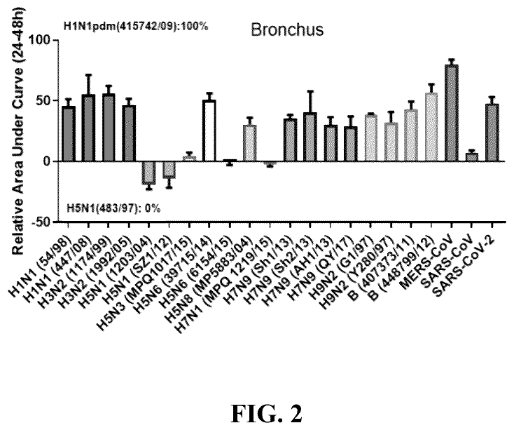

FIG. 2 shows the replication competency of influenza viruses in ex vivo cultures of human bronchus tissues infected with an infectious dose of 10⁶ TCID₅₀/mL at 37° C. The bronchus AUC scores of the tested viruses are presented in Table 2, with scores classified as mild (<25%), moderate (25-74.99%), and high/severe (≥75%).

Referring to FIG. 2 and Table 2, AUC of pandemic H1N1 (415742/97) with high human bronchus infection is set as 100 and H5N1 (483/97) with minimal human bronchus infection is set as 0 for AUC normalization. Seasonal human influenza H1N1 (54/98, 447/08) and H3N2 (1174/99, 1992/05) viruses have around 50% relative AUC with the lowest AUC index of 41.1.

In contrast, HPAI H5N1 (1203/04, SZ1/12) and LPAI surveillance isolates H5N3 (MPQ1017/15), H7N1 (MPQ1219/15) and H5N6 (6154/15) exhibit relative AUC comparable to the control HPAI H5N1 (483/97) virus. Human isolate HPAI H5N6 (39715/14) exhibit markedly higher bronchus replication than avian HPAI H5N6 (6154/15) virus. Interestingly, H7N9 (Sh1/13, Sh2/13, AH1/13, QY/17) and H9N2 (G1/97, Y280/97) exhibit more than 26% relative AUC comparable with some seasonal influenza viruses. MERS-CoV replicates well in human bronchus followed by SARS-CoV-2 but not SARS-CoV.

TABLE 2

| Virus | Bronchus AUC score |
|---|---|
| H1N1 (54/98) | 45.53 |
| H1N1 (447/08) | 55.23 |
| H1N1 (415742/09) | 100.00 |
| H3N2 (1174/99) | 60.21 |
| H3N2 (1992/05) | 41.07 |
| H5N1 (483/97) | 0.00 |
| H5N1 (1203/04) | −19.16 |
| H5N1 (SZ1/12) | −14.12 |
| H5N3 (MPQ1017/15) | −0.18 |
| H5N6 (39715/14) | 57.23 |
| H5N6 (6154/15) | −0.38 |
| H5N8 (MP5883/04) | 30.51 |
| H7N1 (MPQ1219/15) | −8.67 |
| H7N9 (Sh1/13) | 43.45 |
| H7N9 (Sh2/13) | 40.35 |
| H7N9 (AH1/13) | 37.68 |
| H7N9 (QY/17) | 28.77 |
| H9N2 (G1/97) | 43.25 |
| H9N2 (Y280/97) | 26.84 |
| B (407373/11) | 42.97 |
| B (448799/12) | 63.32 |
| MERS-CoV | 79.52 |
| SARS-CoV | −13.03 |
| SARS-CoV-2 | 47.66 |

### Example 6

### Replication Competence of Influenza Viruses and Coronaviruses in Human

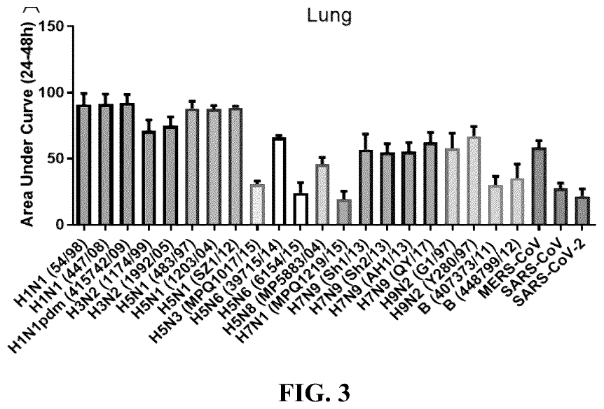

FIG. 3 shows the replication competence of influenza viruses in ex vivo infected human lung tissues, which were exposed to an infectious dose of 10⁶ TCID₅₀/mL at 37° C.

Turning to FIG. 3, most of the viruses replicate efficiently between 24-48 hpi in human lung tissues except H5N6 (6154/15), H7N1 (MPQ1219/15), and SARS-CoV-2 as indicated with mild in the lung AUC score. The lung AUC score of tested viruses is shown in Table 3. Since the lung AUC of H1N1 pandemic is around 100 (97.4 in average), the categorization is established as follows: lung AUC less than 25% is defined as mild, between 25% and 74.99% as moderate, and at or above 75% as high.

TABLE 3
Lung AUC score of tested viruses

| Virus | Lung AUC score |
|---|---|
| H1N1 (54/98) | 82.40 |
| H1N1 (447/08) | 85.42 |
| H1N1 (415742/09) | 97.46 |
| H3N2 (1174/99) | 71.10 |
| H3N2 (1992/05) | 68.72 |
| H5N1 (483/97) | 87.76 |
| H5N1 (1203/04) | 81.69 |
| H5N1 (SZ1/12) | 76.44 |
| H5N3 (MPQ1017/15) | 41.39 |
| H5N6 (39715/14) | 49.14 |
| H5N6 (6154/15) | 24.13 |
| H5N8 (MP5883/04) | 45.88 |
| H7N1 (MPQ1219/15) | 16.27 |
| H7N9 (Sh1/13) | 56.93 |
| H7N9 (Sh2/13) | 54.59 |
| H7N9 (AH1/13) | 55.25 |
| H7N9 (QY/17) | 62.18 |
| H9N2 (G1/97) | 53.58 |
| H9N2 (Y280/97) | 67.10 |
| B (407373/11) | 30.17 |
| B (448799/12) | 35.33 |
| MERS-CoV | 68.66 |
| SARS-CoV | 37.01 |
| SARS-CoV-2 | 21.65 |

Both H1N1 pandemic and HPAI H5N1 replicate efficiently in human lungs despite manifesting different disease severity. Thus, it is apparent that virus replication in the alveolar epithelium is not the key determinant of disease severity.

### Example 7

### Correlation Between Alveolar Fluid Clearance and Severity of Disease in Viruses

In this example, an excellent correlation is discovered between the relative alveolar clearance index with clinically observed severity of disease. All seasonal influenza A (H1N1, H3N2) viruses and influenza B viruses exhibit high relative AFC indices ≥75, as shown in Table 4. All HPAI H5N1 and H7N9 viruses tested, except H5N1 (SZ1/12) and H7N9 (AH1/13) which have around 31 relative AFC, showing severely impaired fluid clearance with a relative AFC of <25. LPAI H5N3 and H7N1 viruses from wild aquatic birds which are not known to cause zoonotic disease as well as H9N2 viruses which cause mild illness in humans, have high relative AFC >65. Interestingly, HPAI H5N8 virus also have high relative AFC. This virus has not caused zoonotic disease but has HPAI H5 HA derived from the A/goose/Guangdong/1/96-like virus lineage, as do H5N1 viruses which cause severe human disease; however, unlike the H5N1 viruses tested, that H5 HA is of clade 2.3.4.4. What is more surprising is that the two HPAI H5N6 viruses have moderate relative AFC between 38-57. These viruses have the H5 HA from clade 2.3.4.4, yet clinically H5N6 viruses do cause severe human disease but infection of children appears to be mild.

TABLE 4
Relative AFC and score of disease severity

In vitro lung injury

| Virus | Relative AFC | Disease severity |
|---|---|---|
| Mock | 100.00 | 0.00 |
| H1N1 (54/98) | 91.35 | 8.65 |
| H1N1 (447/08) | 80.17 | 19.83 |
| H1N1 (415742/09) | 97.47 | 2.53 |
| H3N2 (1174/99) | 82.18 | 17.82 |
| H3N2 (1992/05) | 69.72 | 20.28 |
| H5N1 (483/97) | 6.81 | 93.20 |
| H5N1 (1203/04) | 5.98 | 94.02 |
| H5N1 (SZ1/12) | 27.18 | 72.82 |
| H5N3 (MPQ1017/15) | 78.93 | 21.07 |
| H5N6 (39715/14) | 37.93 | 62.07 |
| H5N6 (6154/15) | 56.79 | 43.21 |
| H5N8 (MP5883/04) | 63.03 | 36.97 |
| H7N1 (MPQ1219/15) | 74.09 | 25.91 |
| H7N9 (Sh1/13) | 18.99 | 81.01 |
| H7N9 (Sh2/13) | 11.93 | 88.07 |
| H7N9 (AH1/13) | 30.57 | 69.43 |
| H7N9 (QY/17) | 9.97 | 90.03 |
| H9N2 (G1/97) | 65.87 | 34.13 |
| H9N2 (Y280/97) | 68.41 | 31.59 |
| B (407373/11) | 94.66 | 5.34 |
| B (448799/12) | 79.34 | 20.66 |
| MERS-CoV | 8.14 | 91.86 |
| SARS-CoV | 6.80 | 93.20 |
| SARS-CoV-2 | 67.81 | 32.19 |

Table 4 summarizes the relative AFC and score of disease severity of the tested viruses. The scores are derived from the results obtained using the lung injury model of the tested viruses. <25% as mild, 25-74.99% as moderate and 75% as severe.

Table 5 summarizes the overall scores of the tested viruses including the risk of human transmission based on the score of bronchus AUC, risk of lung infection based on the score of lung AUC and disease severity based on the relative AFC induced by the tested viruses.

TABLE 5
Virus overall score

| | | | In vitro |
|---|---|---|---|
| | Ex vivo model | | lung injury |
| | Risk of human | Risk of lung | Disease |
| Virus | transmission | infection | severity |
| H1N1 (54/98) | Moderate | High | Mild |
| H1N1 (447/08) | Moderate | High | Mild |
| H1N1 (415742/09) | High | High | Mild |
| H3N2 (1174/99) | Moderate | Moderate | Mild |
| H3N2 (1992/05) | Moderate | Moderate | Mild |
| H5N1 (483/97) | Mild | High | Severe |
| H5N1 (1203/04) | Mild | High | Severe |
| H5N1 (SZ1/12) | Mild | High | Moderate |
| H5N3 (MPQ1017/15) | Mild | Moderate | Mild |
| H5N6 (39715/14) | Moderate | Moderate | Moderate |
| H5N6 (6154/15) | Mild | Mild | Moderate |
| H5N8 (MP5883/04) | Moderate | Moderate | Moderate |
| H7N1 (MPQ1219/15) | Mild | Mild | Moderate |
| H7N9 (Sh1/13) | Moderate | Moderate | Severe |
| H7N9 (Sh2/13) | Moderate | Moderate | Severe |
| H7N9 (AH1/13) | Moderate | Moderate | Moderate |
| H7N9 (QY/17) | Moderate | Moderate | Severe |
| H9N2 (G1/97) | Moderate | Moderate | Moderate |
| H9N2(Y280/97) | Moderate | Moderate | Moderate |
| B (407373/11) | Moderate | Moderate | Mild |
| B (448799/12) | Moderate | Moderate | Mild |
| MERS-CoV | High | Moderate | Severe |
| SARS-CoV | Mild | Moderate | Severe |
| SARS-CoV-2 | Moderate | Mild | Moderate |

### Example 8

Comparison with Traditional Methods in Assessing Viral Transmission and Disease Severity

The present dual-system method is not included in neither of CDC's IRAT nor WHO's TIPRA. It is the only system using human tissues and acute lung injury model for assessment of both transmission and disease severity induced by viruses infecting human respiratory tissues and this approach is irrespective of the virus ability to productively replicate in the human respiratory tissues. There is no such dual-system method approach using ex vivo human respiratory tissues published. Therefore, regarding the IRAT or TIPRA, which consists of 10 different elements, our innovation described here provides valuable insight and superior enhancement in the accuracy of disease severity determination, which can be adopted in and contribute to also the enhancement in the accuracy of the scoring system in IRAT and TIPRA.

In summary, the present invention provides novel methods for improving pandemic risk assessment of animal influenza viruses that can be used to advance current risk assessment algorithms. By using two reference viruses representing a virus with known pandemic potential (i.e. the 2009 pandemic H1N1 virus) and one that has been zoonotic for over two decades without acquiring transmissibility between humans (i.e. HPAI H5N1) as two polar ends of the spectrum for bronchus tropism and using mock infection as 100% AFC for relative AFC analyses, a more reproducible and semi-quantifiable approach is now available. This method utilizes data from ex vivo cultures of human bronchus for pandemic risk assessment of animal influenza viruses.

Since acute lung injury is a pathogenic pathway for severe influenza disease, the in vitro acute lung injury model was utilized to assess the impact of each virus on AFC. It is also the first study to explore the ability of coronaviruses to affect fluid transport in vitro. Interestingly, there was moderately negative correlation between viral replication in AECs and relative AFC index in the transwell cultures. HPAI H5N1 viruses have high viral replication (indicated by AUC) and low relative AFC which gave rise to a negative correlation.

However, pandemic H1N1 with moderate viral replication and high relative AFC has little or moderate correlation. This supports the statement that acute lung injury is not determined by virus tropism on the lung alveolar epithelium alone, but is more related to the nature of mediators released from these cells.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

The term “dual-system method” refers to an integrated approach that utilizes two distinct yet complementary systems to evaluate the human transmissibility and disease severity of respiratory viruses. This method combines ex vivo human airway cultures and an in vitro lung injury model. Specifically, the ex vivo airway cultures are used to assess the viral replication competence of respiratory viruses in bronchial and lung tissues, while the in vitro lung injury model is employed to evaluate alveolar fluid clearance AFC impairment, which is a critical determinant of transmission risk. By incorporating both systems, this approach allows for a more comprehensive assessment of viral behavior and its implications for disease severity and transmissibility, offering advantages over traditional single-system models that may lack the complexity and physiological relevance required for accurate results.

The term “in vitro lung injury model” is a laboratory-based system that mimics the conditions of lung injury by using primary human AECs cultured on apical Transwell inserts. This model is designed to simulate AFC, a key physiological process in the lungs that can be impaired during viral infection. By utilizing this in vitro setup, the model enables researchers to evaluate the impact of respiratory viruses on AFC, providing insight into the degree of lung injury and transmission risk. The in vitro lung injury model serves as a valuable tool for studying the effects of viral infections on alveolar function, as it closely replicates the epithelial barrier and fluid transport properties of human lungs, offering a more controlled environment for assessing viral-induced damage.

The term “Alveolar fluid clearance (AFC)” refers to a physiological process by which fluid is transported from the alveolar space into the interstitial and vascular compartments of the lung. This process is essential for maintaining proper lung function and preventing the accumulation of excess fluid in the alveoli, which could impair gas exchange and contribute to respiratory failure. In healthy individuals, AFC is regulated by the action of alveolar epithelial cells, primarily through the movement of sodium ions and water across the epithelial barrier. During viral infections, however, AFC can be disrupted, leading to fluid retention and worsening of lung injury. As such, assessing AFC impairment in response to respiratory viruses is an important indicator of disease severity and can help predict the risk of transmission and the overall impact of the infection on lung function.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

What is claimed is:

1. A dual-system method for assessing the human transmissibility and disease severity of respiratory viruses, comprising:
utilizing ex vivo human airway cultures to evaluate viral replication competence of the respiratory viruses in bronchial and lung tissues;
utilizing an in vitro lung injury model to evaluate alveolar fluid clearance (AFC) impairment induced by the respiratory viruses to determine disease severity; and
correlating the viral replication competence in the bronchial and lung tissues with relative human transmissibility score.

2. The method of claim 1, wherein the ex vivo human airway cultures comprise human bronchial tissues obtained from non-malignant lung resection surgeries.

3. The method of claim 1, wherein the viral replication competence is assessed by measuring viral titers in the culture supernatant at 1, 24, and 48 hours post-infection using a median tissue culture infectious dose assay.

4. The method of claim 1, further comprising normalizing area under the curve (AUC) of viral replication kinetic curves to reference strains to determine the relative human transmissibility score.

5. The method of claim 4, wherein the reference strains comprise pandemic influenza A subtype H1N1 and highly pathogenic avian influenza A subtype H5N1.

6. The method of claim 1, wherein the in vitro lung injury model utilizes primary human alveolar epithelial cells (AECs) cultured on apical Transwell inserts to simulate the AFC.

7. The method of claim 1, wherein the AFC impairment is quantified by measuring changes in fluorescent intensity over a 24-hour infection period, and the AFC is calculated relative to mock-infected controls, with mock infections set as 100% AFC.

8. The method of claim 1, wherein the respiratory viruses comprise influenza A viruses, influenza B viruses, respiratory syncytial virus (RSV), adenoviruses, parainfluenza viruses, rhinoviruses, human metapneumovirus, enterovirus, or coronaviruses.

9. The method of claim 8, wherein the influenza A viruses include HIN1, H3N2, H5N1, H5N6, H5N8, H7N9, and H9N2 subtypes.

10. The method of claim 8, wherein the coronaviruses include SARS-CoV, MERS-CoV, and SARS-CoV-2.

11. The method of claim 1, further comprising comparing the AFC impairment of the respiratory virus to that of reference strains to categorize disease severity.

12. The method of claim 1, further comprising evaluating the impact of varying multiplicities of infection (MOIs) on the AFC impairment to assess dose-dependent pathogenicity.

13. The method of claim 12, wherein the MOIs range from 0.1 to 10.

14. The method of claim 13, wherein the representative MOIs is approximately 0.1.

15. The method of claim 1, wherein the AFC impairment is attributed to virus-induced soluble mediators rather than direct cytopathic effects.

16. The method of claim 1, further comprising calculating a relative disease severity score by comparing AFC impairment with reference treatment and strains.

17. The method of claim 1, wherein the dual-system method exhibits improved accuracy of quantification in disease severity compared to traditional ex vivo human airway cultures, showing 65.2%, 20.6% and 23.6% improvement in quantification accuracy in disease severity of seasonal influenza, avian influenza, and low pathogenic avian and influenza B viruses, respectively.
